(19) United States
(12) Patent Application Publication
(10) Pub. No.: US 2026/0198831 A1
(43) Pub. Date: Jul. 16, 2026

(54) ACTIVE ARRAY FOR HIGH-FIDELITY ACTIVE BRAIN CORTEX SIGNAL MEASUREMENT DEVICE

(71) Applicant: INDUSTRY-ACADEMIC COOPERATION FOUNDATION, YONSEI UNIVERSITY, Seoul (KR)

(72) Inventors: Jong Hyun AHN, Seoul (KR); Duo XU, Seoul (KR); Ju Yeong HONG, Seoul (KR)

(21) Appl. No.: 19/406,246

(22) Filed: Dec. 2, 2025

(30) Foreign Application Priority Data
Dec. 9, 2024 (KR) 10-2024-0181567

(51) Int. Cl. A61B 5/293; A61B 5/00; A61B 5/263

(52) U.S. Cl. CPC A61B 5/293; A61B 5/263; A61B 5/6868; A61B 2562/046; A61B 2562/125

(57) ABSTRACT

According to an embodiment, a two-dimensional molybdenum disulfide-based flexible active matrix method for ECoG monitoring that implements ultra-high-resolution spatiotemporal mapping of various brain signals can be obtained.

### CROSS REFERENCE TO RELATED APPLICATION

The present application claims priority to Korean Patent Application No. 10-2024-0181567, filed on Dec. 9, 2024, the entire contents of which are incorporated here for all purposes by this reference.

### TECHNICAL FIELD

The present invention relates to an active array for high-resolution active cortical signal measurement devices, and more particularly, to a flexible two-dimensional (2D) molybdenum disulfide (MoS₂)-based active array for high-resolution electrocorticography (ECoG) monitoring of bioelectrical signals.

### BACKGROUND ART

Over the past decades, significant advances in neurotechnology, particularly in the field of neurophysiological monitoring, have enabled an unprecedented and comprehensive understanding of brain functions. Based on these advancements, it is expected that major challenges related to brain activity will be addressed, including the diagnosis and monitoring of various neurological disorders (e.g., epilepsy, attention deficit hyperactivity disorder (ADHD), tinnitus, stroke, dementia, and Parkinson's disease), as well as the extraction and decoding of characteristics of various brain activities (e.g., sensorimotor control, speech and language identification, and stimulus-sensory information processing).

A profound understanding of neural activity depends on the development of an ideal neural interface. This interface must be capable of monitoring vast populations of neurons with exceptional spatiotemporal resolution. However, in the face of inherent limitations in spatiotemporal coverage and data processing throughput, finding a method to harmonize high resolution with signal fidelity remains a critical challenge.

Several methods exist for providing valuable insights into neural function by recording the electrical activity of the brain. Electrocorticography (ECoG) measurement, which involves placing an electrode array directly on the cortical surface, is emerging as a rapidly growing research field due to its minimally invasive nature and its ability to record high-quality signals in response to sensorimotor stimuli.

ECoG electrodes are classified into two types: passive arrays and active arrays. Passive arrays, consisting only of electrodes and an insulating layer, have a simple design and allow for the direct readout of membrane potentials; however, they face limitations in electrode density, signal fidelity, and separation when the number of electrodes needs to be scaled. In contrast, active arrays integrate transistors for multiplexing and readout to reduce the number of interconnections. By integrating transistors in juxtaposition with sensing electrodes, active arrays can increase electrode density, accelerate data throughput, and maintain stable signal fidelity. Nevertheless, finding universally compatible materials and designs for such flexible active arrays remains a significant challenge in current research.

Various semiconductor materials have been developed for active ECoG measurements. Organic materials are excellent in terms of large-scale manufacturing, biodegradability, and flexibility. However, they struggle with high-fidelity recording at high multiplexing densities and face workloads limited by mobility and operating voltage. Inorganic materials, such as silicon (Si) nanomembranes, offer superior electrical properties but require complex transfer processes and have limited mechanical flexibility. Graphene is highly conductive but prone to leakage and vulnerable to noise in complex active arrays due to its zero-band gap.

### PRIOR ART DOCUMENTS

### Patent Documents

(Patent Document 0001) Korean Unexamined Patent Publication No. 10-2018-0066620 (Published on Jun. 19, 2018)

### SUMMARY OF INVENTION

### Technical Problem

An object of the present invention is to provide an active matrix scheme for high-resolution electrocorticography (ECoG) monitoring of bioelectrical signals.

Another object of the present invention is to provide an active matrix scheme for ECoG monitoring that allows for functional coordination between active multiplexing and sensing.

### Solution to Problem

According to an aspect of the present invention, there is provided an active array for a high-resolution active cortical signal measurement device, comprising a unit pixel which includes a sensing thin-film transistor (TFT) for sensing brain signals of a living body and a multiplexing TFT connected in series with the sensing TFT for multiplexing.

According to another aspect of the present invention, there is provided a method of manufacturing an active array for a high-resolution active cortical signal measurement device, the method comprising the steps of: (S100) depositing a buffer layer on a polymer substrate; (S200) forming a two-dimensional (2D) transition metal dichalcogenide channel on the buffer layer; (S300) forming a source electrode and a drain electrode on the 2D transition metal dichalcogenide channel; (S400) forming a gate dielectric layer on the source electrode and the drain electrode; (S500) forming a gate electrode on the gate dielectric layer; and (S600) forming an encapsulation layer on the gate electrode.

### Advantageous Effects of Invention

According to an embodiment of the present invention, it is possible to obtain a flexible active matrix scheme based on two-dimensional (2D) molybdenum disulfide for ECoG monitoring that implements ultra-high resolution spatiotemporal mapping of various brain signals.

According to another embodiment of the present invention, functional coordination between active multiplexing and sensing is enabled by connecting two molybdenum disulfide thin-film transistors (TFTs) in series.

### BRIEF DESCRIPTION OF DRAWINGS

FIGS. 1A-1F illustrate a development process of an active-type multiplexing MoS2 array for neural sensing according to an embodiment of the present invention.

FIGS. 2A-2I illustrate electrical characteristics of a dual-TFT pixel of an $MoS_2$-based active array according to an embodiment of the present invention.

FIGS. 3A-3I illustrate in vitro recordings of normal and epileptic activities with high spatiotemporal resolution according to an embodiment of the present invention.

FIGS. 4A-4G illustrate in vivo monitoring of event-related potentials (ERP) in the auditory cortex evoked by acoustic stimulation according to an embodiment of the present invention.

### DETAILED DESCRIPTION OF THE INVENTION

The present inventor has analyzed the problems of conventional active arrays for ECoG measurement and conducted intensive research, leading to the derivation of the following invention.

Specifically, the present disclosure provides an MoS2-based active array that achieves high-resolution spatiotemporal mapping of ECoG signals, as demonstrated by the successful mapping of sensory stimuli, sound-evoked tonotopic maps, and event-related potentials (ERPs) for epileptiform activity in in vitro brain models. By directly growing a 4-inch trilayer low-temperature MoS2 on a polyimide (PI) substrate, eight 10×10 active arrays could be simultaneously fabricated. Utilizing multiplexing and monolithic integration, the scalable MoS2 multichannel array concurrently sampled brain signals at an impressive pixel density of 16 pixels per millimeter in in vivo mouse experiments using implantable devices. The active array exhibited high temporal resolution at a sub-millisecond sampling level, and consequently, accurately evaluated epileptiform ECoG signals with a correlation of 94.4% in in vitro experiments. Furthermore, the MoS2 array showed remarkably high fidelity and resolution when recording auditory cortex activity, which was validated through spatiotemporal mapping of pure-tone ERP, auditory mismatch negativity (AMMN), and tonotopic organization, offering vast potential for accessing sensory responses to stimuli.

According to an aspect of the present invention, an active array for a high-resolution active cortical signal measurement device may include a unit pixel comprising a sensing TFT for sensing brain signals of a living body, and a multiplexing TFT connected in series with the sensing TFT for multiplexing.

According to an embodiment of the present invention, the sensing TFT and the multiplexing TFT may comprise two-dimensional (2D) molybdenum disulfide (MoS2) thin-film transistors. 2D molybdenum disulfide (MoS2) is an ideal material for ECoG active arrays due to its excellent electrical and mechanical properties, biocompatibility, and monolithic fabrication. MoS2 exhibits high mobility (>10 cm²V⁻¹s⁻¹), a significant on/off ratio (~10⁸), and a low operating voltage (<10 V), thereby enabling high signal fidelity and rapid switching characteristics across various frequencies. These properties overcome most of the disadvantages of conventional semiconductor materials. In addition, the MoS2-based active array, with a fracturing strain level of ~3.5%, can be seamlessly integrated into the cerebral cortex due to its flexible and conformal characteristics without signal loss or tissue damage.

According to an embodiment of the present invention, the column size of the unit pixel may be 250 μm or less. According to another embodiment, the resolution in the active array may be 16 pixels/mm² or more.

According to another aspect of the present invention, a method of manufacturing an active array for a high-resolution active cortical signal measurement device may include the steps of: (S100) depositing a buffer layer on a polymer substrate; (S200) forming a 2D transition metal dichalcogenide (TMDC) channel on the buffer layer; (S300) forming a source electrode and a drain electrode on the 2D TMDC; (S400) forming a gate dielectric layer on the source electrode and the drain electrode; (S500) forming a gate electrode on the gate dielectric layer; and (S600) forming an encapsulation layer on the gate electrode.

According to an embodiment of the present invention, the 2D TMDC may be at least one selected from the group consisting of MoS2, WSe2, WS2, and MoTe2.

According to an embodiment of the present invention, the step (S200) of forming the 2D TMDC channel on the buffer layer may include a step (S210) of directly growing the 2D TMDC on the buffer layer at a temperature ranging from 100° C. to 400° C. Outside this range, if the temperature is too high, disadvantages such as damage to wiring (copper, gold) due to oxidation and combustion of the polymer thin film may occur. Conversely, if the temperature is too low, it is difficult for the precursor reaction to occur, making uniform thin-film growth impossible.

In other words, unlike graphene or silicon-based devices, the low-temperature (LT) MoS2 according to an embodiment of the present invention allows for wafer-scale direct fabrication through monolithic integration on a polymer substrate. This reduces fabrication steps and interface contamination, thereby improving the manufacturing yield of flexible neural interfaces.

According to an embodiment of the present invention, the 2D TMDC is 2D molybdenum disulfide (MoS2), and the step (S210) of directly growing the 2D MoS2 on the buffer layer at a temperature of 100° C. to 400° C. may utilize molybdenum hexacarbonyl (MHC) and anhydrous dimethyl sulfide (DMS) as precursors. These may be provided to a metal-organic chemical vapor deposition (MOCVD) system in the form of a mixed precursor solution.

According to another embodiment, the step (S200) of forming the 2D TMDC channel on the buffer layer may include a step (S220) of growing the 2D TMDC at a temperature exceeding 400° C. and then transferring it onto the buffer layer.

Hereinafter, the present invention will be described in more detail through examples. The following examples are merely illustrative to aid understanding of the present invention, and the scope of the present invention is not limited thereto.

### EXAMPLES

### 1. Low-Temperature MoS2 Synthesis

To obtain uniform and high-quality 2 D MoS2 on a 4-inch wafer, a customized three-zone MOCVD system for low-temperature MoS2 growth was utilized, employing molybdenum hexacarbonyl (MHC, Sigma-Aldrich, 577766, ≥99.9%) and anhydrous dimethyl sulfide (DMS, Sigma-Aldrich, 274380, ≥99.0%) as precursors. A mixed precursor solution, specifically 68 mg of MHC dissolved in 15 mL of DMS, facilitated controlled source injection into the chamber. The substrate was placed in the second zone of the MOCVD chamber, which was maintained at 150° C. and 6 Torr via an automatic pressure controller (MKS 600 series). A constant mixed carrier gas of 800 sccm Ar and 3 sccm H2 was established and maintained throughout the entire process. The synthesis of few-layer MoS2 began with 0.7 sccm Ar to bubble the mixture (MHC+DMS) during the first hour (nucleation stage), followed by an increased flow rate of 2.7 sccm for the subsequent 33 hours (growth stage).

### 2. Fabrication of MoS2 Array

Prior to fabricating the TFT array, a flexible polyimide (PI, Sigma-Aldrich, 15 wt. %) substrate was pre-spin-coated (10 μm) onto a rigid substrate (quartz), and a 10 nm Al2O3 buffer layer was deposited on the PI substrate via an atomic layer deposition (ALD) process. A thin-film transistor (TFT) array was constructed by monolithically integrating two layers of metal electrodes/interconnects and one Al2O3 layer onto the patterned 2D MoS2 channel layer. To create top-gate type TFTs, the MoS2 channel was patterned, followed by the fabrication of source/drain electrodes (Cr/Au, 4/100 nm) with a channel length (L)/width (W) of 8/200μm through photolithography (photoresist AZ nLOF 2035), thermal evaporation, and lift-off processes. After depositing a 30 nm Al2O3 layer as a gate dielectric using a 150° C. ALD process, the top-gate electrodes (Cr/Au, 4/100 nm) were deposited. Subsequently, the PI substrate was patterned using reactive ion etching (RIE) with plasma (SF6:O2=10 sccm: 50 sccm, 1200 seconds) to match the exposed area of a mouse's craniotomy. Finally, for an ideal device-tissue interface, an SU-8 (Sigma-Aldrich) encapsulation layer (2 μm) was formed to protect the device, leaving only the sensing pads exposed via photolithography.

### 3. Device Characterization

The electrical characteristics of the dual-TFT unit were characterized using a four-probe platform (SCS-4200) under a drain-source bias ($V_{ds}$) of 0.1 V. To verify switching performance, the switching characteristics of the entire pixel were inspected while the internal sensing TFT was activated. For this purpose, the two gate electrodes of the unit cell's TFTs were separately connected to a switching control circuit and a ground line (GND, 0 V), respectively.

### 4. ROC Design

A custom Readout Circuit (ROC) was designed for data acquisition. The ROC consisted of a transimpedance circuit for voltage-mode conversion, a switching module (Panasonic, AQY221N 5) for multiplexing, a band-pass filter (0.1 Hz to 2000 Hz), and a 1 MSPS analog-to-digital converter (Analog Devices, LTC2368) integrated with a Bluetooth Low Energy (BLE) System-on-Chip (SoC) for wireless data transmission. After digitization, the data was automatically calibrated according to a transmission curve and then transmitted.

### 5. Animal and Surgical Procedures

All animal handling procedures were approved by the Institutional Animal Care and Use Committee (IACUC, A-0117) of the City University of Hong Kong. This study complied with all requirements specified in the institutional guidelines of the Animal Welfare Committee. Mice were housed in a temperature-controlled environment (23° C.) under a 12-hour light/dark cycle, with ad libitum access to food and water. A 12-week-old male C57BL/6J mouse was anesthetized via intraperitoneal (IP) injection of ketamine (120 mg/kg) and xylazine (12 mg/kg) based on body weight. Additional IP doses of one-third of the initial dose were administered as needed. The mouse was prepared for cortical recording as follows: first, the mouse was placed in a stereotaxic apparatus to fix the head position. Throughout the anesthesia, body temperature (~36.5° C.) and physiological status (respiratory rate, heart rate, corneal reflex, and hind-paw withdrawal reflex) were continuously monitored. Next, to reduce the possibility of cerebral edema, cerebrospinal fluid (CSF) was drained through the dura mater, and a craniotomy was performed on the temporal cortex to expose the primary auditory cortex (A1) of one hemisphere via a dural incision. Subsequently, the MoS2 array was applied to the surface of the cerebral cortex to record neural activity.

### 6. Data Acquisition

Neural activity responding to acoustic stimuli (i.e., local field potentials; LFPs) was recorded from the cortical surface of the A1 using the MoS2-based electrode array. Acoustic stimuli were delivered via a close-field MF1 speaker (Tucker-Davis Technologies). To record pure tone-evoked LFPs, 50-ms pure tones (8 or 30 kHz) were presented every 3 seconds at a sound pressure level (SPL) of 75 dB, with each frequency played individually for at least 1 minute. The auditory mapping procedure is described in detail elsewhere. To reconstruct the frequency-intensity receptive fields (RFs) for auditory mapping, 50-ms tone pips (equipped with a 2-ms cosine-squared ramp) at 80 different frequencies (4-32 kHz with 0.1-octave intervals) and 8 SPLs (0-70 dB in 10-dB steps) were played pseudo-randomly every 0.5 seconds, presented repetitively for three iterations each.

### 7. Data Analysis

All neurophysiological data were processed and analyzed using licensed MATLAB R2023a and its built-in Signal Analyzer toolbox, where the recorded signals were sampled at 2 kHz and filtered using band-stop and high-pass (>0.1 Hz) filtering to improve quality and visibility. For auditory mapping, sound-evoked spikes were detected from LFPs using a custom script based on an algorithm adapted from Ko³odziej et al. (2018), and the raw LFP data were downsampled to 1 kHz and filtered with a band-pass (0.5-100 Hz) Butterworth filter to effectively extract cortical sound-evoked response activities and remove artifacts. Spike candidates were selected by matching the filtered data with a triangle-shaped pattern within a sliding window (41 ms) that operated from 10 ms to 30 ms after the onset of the sound stimulus at 2-ms intervals. A segment was considered a spike if it met or exceeded two predefined thresholds: (1) an absolute Pearson correlation coefficient threshold of 0.7 between the pattern and the data within the window, and (2) an amplitude threshold of 2.5 times the root mean square (RMS) of the corresponding channel data in the detected segment. Subsequently, frequency-intensity response maps for all channels were obtained along with the detected spikes, and the maps were first smoothed by applying an average 3×3 filter. Afterward, a V-shaped RF was manually determined for each map, and channels with sparse spikes that failed to form a V-shaped curve were excluded when decoding the characteristic frequency (CF). The CF was determined as the frequency that induced a response at the lowest sound intensity, typically corresponding to the tip of the V-shaped curve, and in cases where multiple frequency candidates existed at the lowest sound intensity, the candidate with the highest total number of spikes summed across all sound intensities was determined as the CF, and finally, the cortical tonotopic map was reconstructed using the decoded CFs.

### Experimental Examples

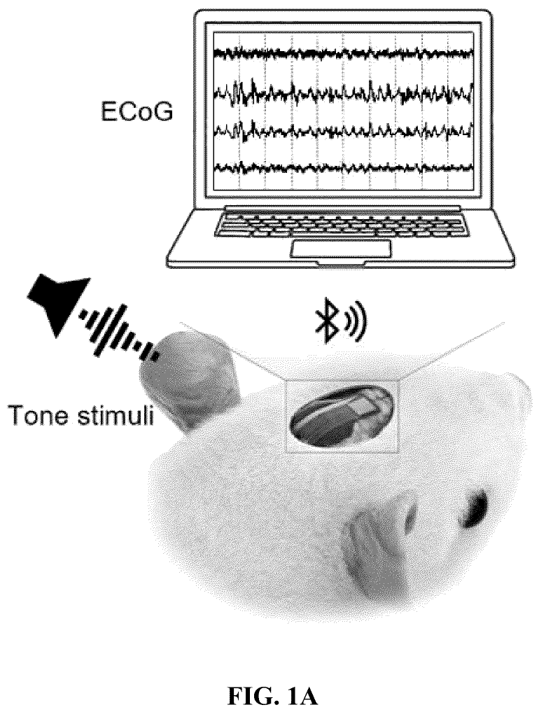

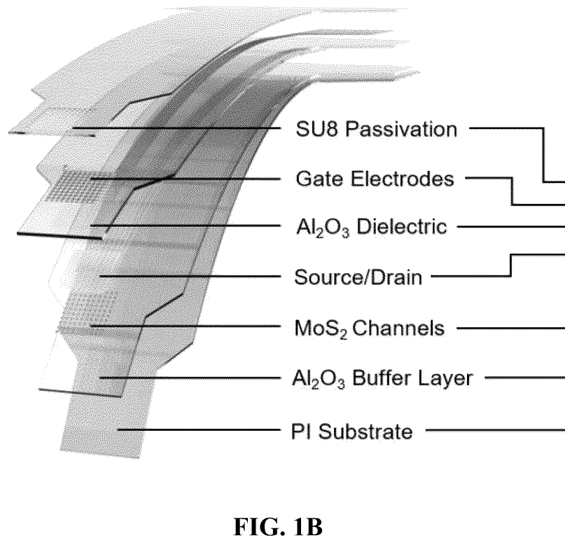

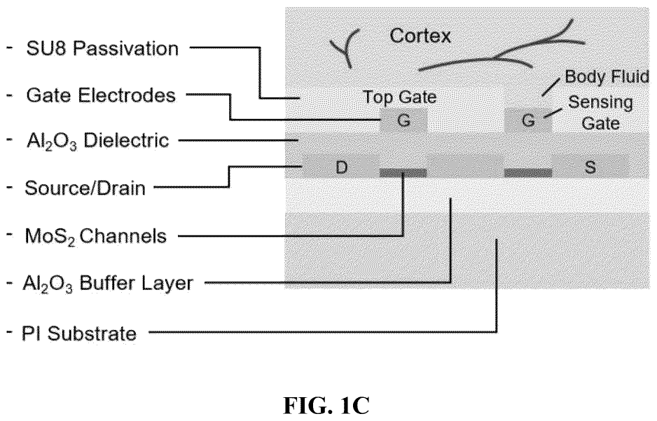

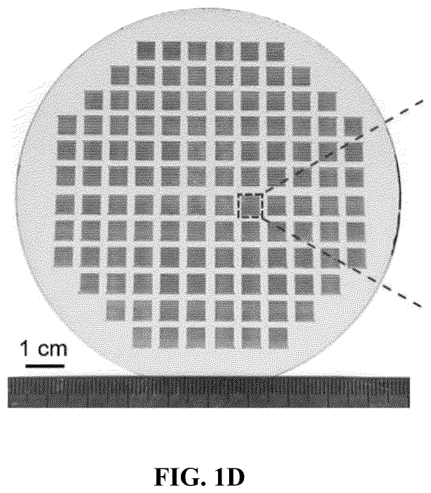

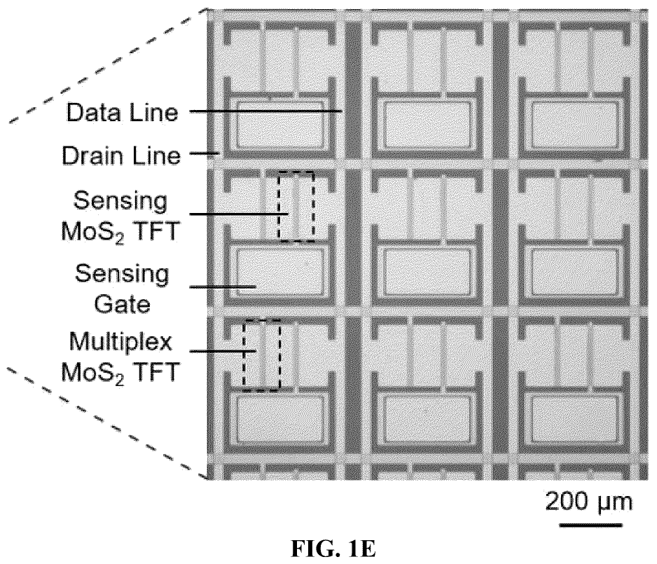

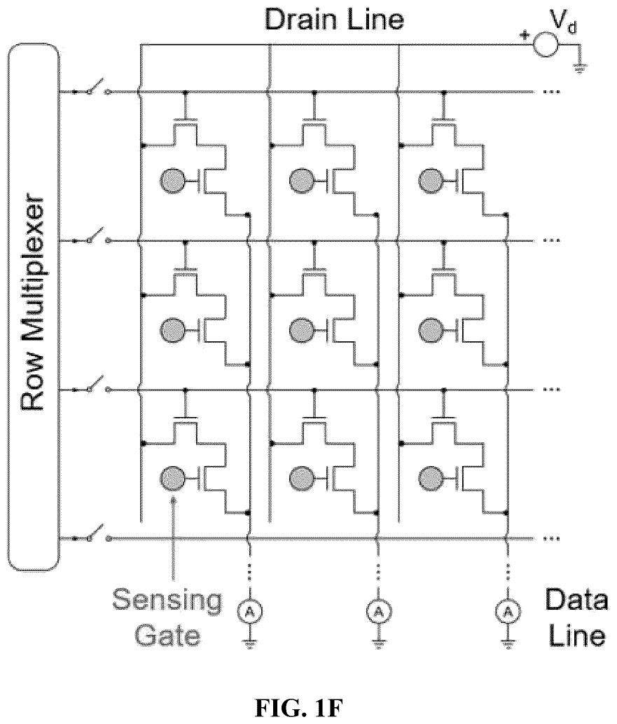

FIG. 1 illustrates a development process of an active-type multiplexing MoS2 array for neural sensing according to an embodiment of the present invention.

Specifically, FIG. 1a is a schematic diagram of an implantable MoS2-based ECoG sensing system for high-fidelity recording of auditory responses, FIG. 1b is an exploded view of an MoS2-based active array highlighting key functional layers of a top-gate type TFT array structure, FIG. 1c is a schematic cross-sectional view of a single pixel of an active array with built-in (2T) multiplexing and sensing TFT units in series, FIG. 1d is a photograph of an MoS2-based active matrix fabricated on a 4-inch wafer scale on a PI substrate through monolithic integration, FIG. 1e is an optical illustration of a single active matrix, where pixels share data lines and drain lines, connected to a time-domain multiplexing (TDM) and readout circuit, and FIG. 1f is a schematic diagram of the operating principle of a multiplexing circuit for an active array integrated with an ammeter readout circuit for each column for row addressing.

Referring to FIG. 1a, the design and fabrication of the active array were based on 4-inch low-temperature grown MoS2 using a metal-organic chemical vapor deposition (MOCVD) method, where a top-gated thin-film transistor (TFT)-based array was integrated for active ECoG recording. By omitting the transfer process generally present in silicon-based flexible device fabrication through monolithic integration on directly synthesized MoS2, interface contamination and mechanical damage could be reduced.

Referring to FIG. 1b, Al2O3 served as a top-gate dielectric medium for current-voltage modulation of the sensing MoS2 TFT and as an interlayer dielectric for the interconnections of the array. On the backside of the device, a single buffer layer (40 nm Al2O3) was pre-prepared on the PI substrate to provide a low-temperature growth interface for MoS2 and to prevent mobility degradation due to surface roughness scattering of the polymer. On the frontside, SU8-based encapsulation was utilized as a biofluid barrier, leaving window patterns only at the locations of the Au sensing pads.

Referring to FIG. 1c, the SU8 layer also provided adequate support during device implantation, preventing wrinkling of the conformal electrode-cortical interface.

Referring to FIG. 1d, the fabricated matrix is shown, verifying the fabrication of a complete MoS2-based active array with interconnections on a 4-inch PI/sapphire substrate, yielding a total of 4,000 n-type metal-oxide-semiconductor field-effect transistors (MOSFETs) consisting of eight 10×10 sensor arrays and 12 MoS2 test modules through monolithic integration.

Referring to FIG. 1e, in the active matrix, each pixel was configured in series with two independent MoS2 TFTs to coordinate the functions of integrated multiplexing and ECoG sensing. While the multiplexing TFT allowed pixels to share drain bias and data lines, effectively minimizing interconnections compared to passive arrays, the sensing TFT integrated in a source-follower configuration implemented high input impedance and reduced the physical distance between electrodes and readout components, thereby enabling the elimination of crosstalk and noise.

Therefore, referring to FIG. 1f, a trans-impedance, time-domain multiplexing (TDM) readout circuit (ROC) was designed to facilitate rapid row-wise switching of pixels and to acquire current-mode signals from the active array.

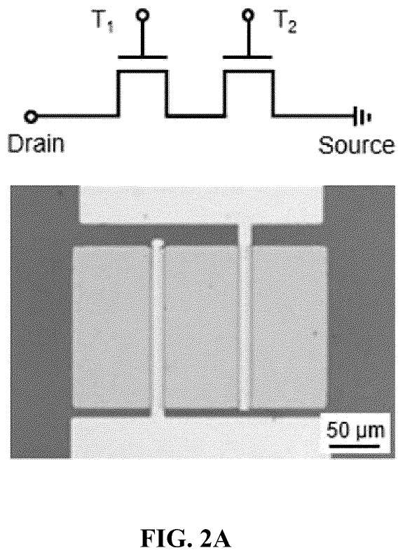

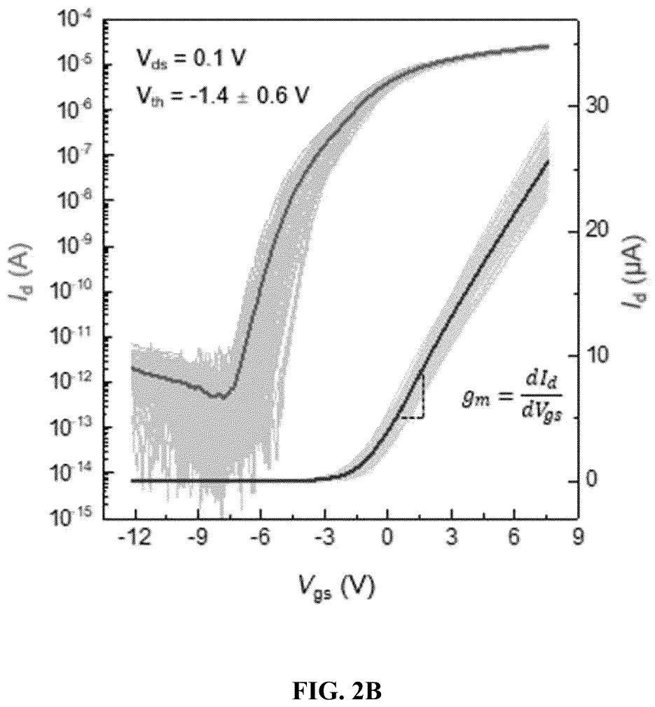

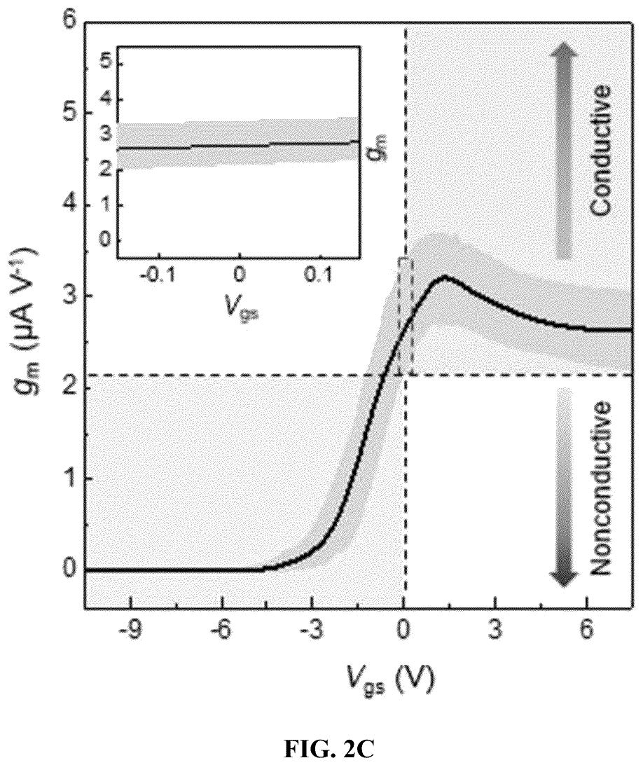

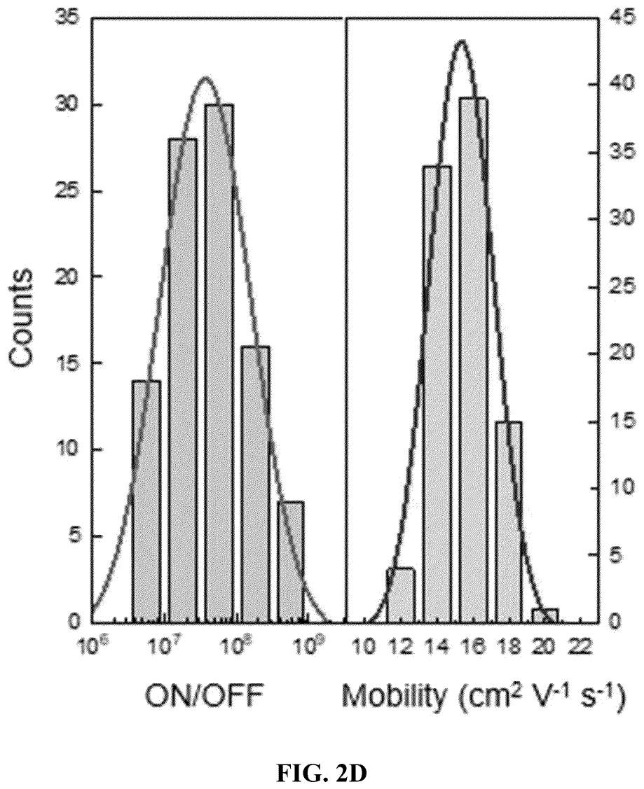

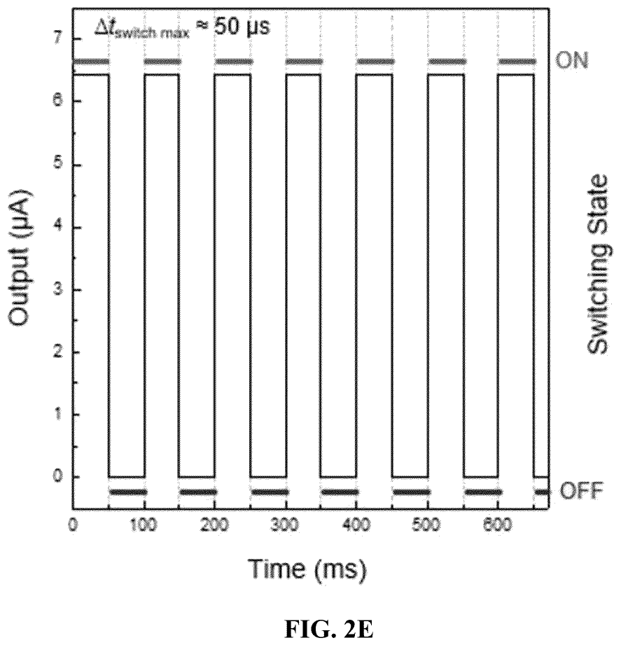

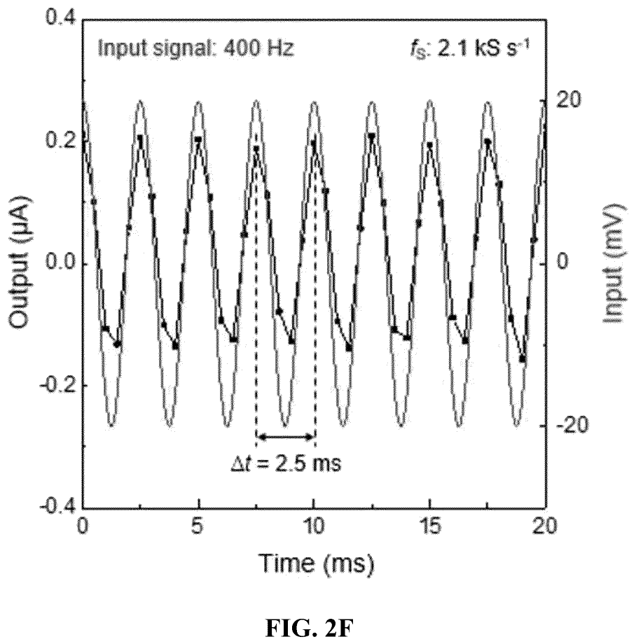

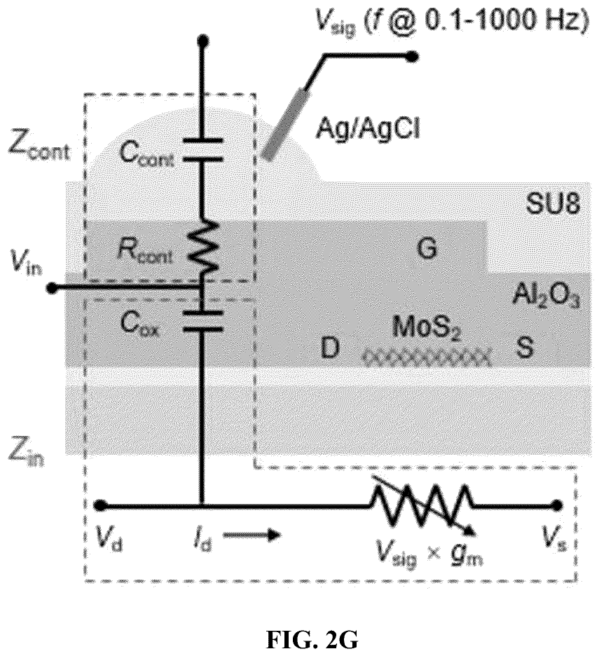

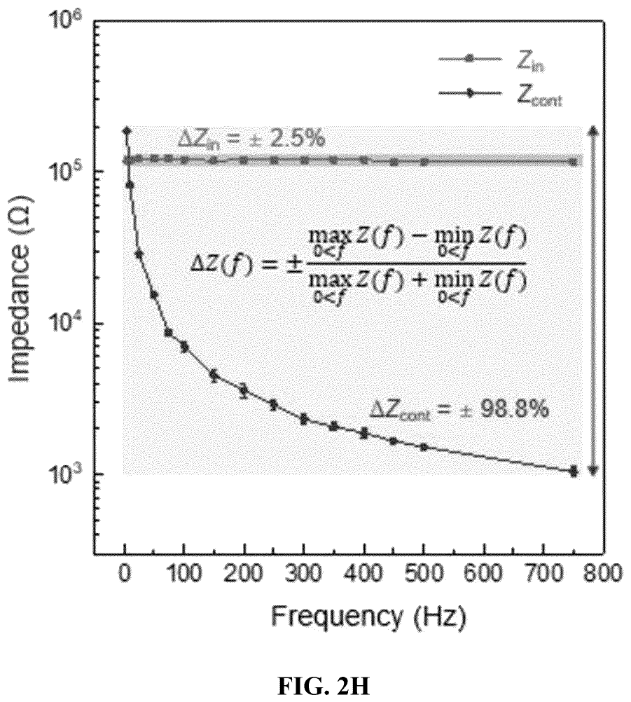

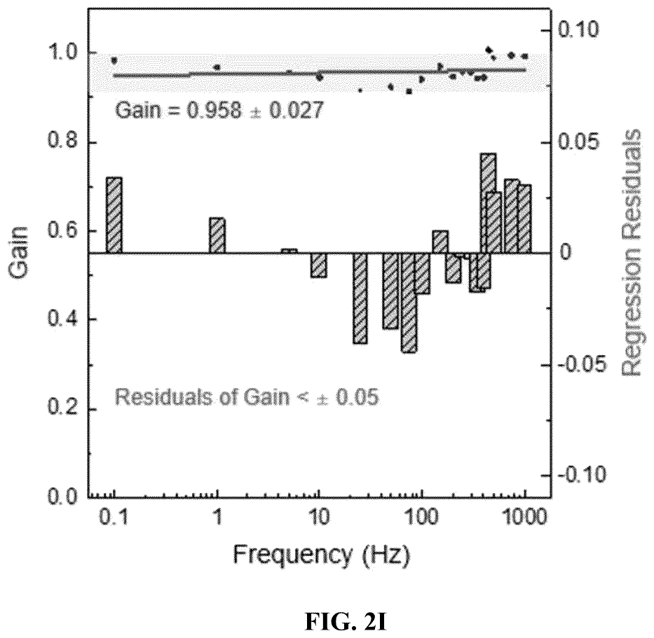

FIG. 2 shows the electrical characteristics of a dual-TFT pixel of an MoS2-based active array according to an embodiment of the present invention.

Specifically, FIG. 2a is a photograph and a schematic circuit diagram of a single array pixel with two TFTs in a series winding structure, FIG. 2b is the I-V transfer characteristics of the MoS2 TFT at Vds=0.1 V, where fully synchronized voltage sweeping is established at two gate terminals (T1 and T2) through a four-probe platform, FIG. 2c is the gm-Vgs relationship of the depletion-mode MoS2 TFT, showing the conductive state of the MoS2 channel at a proximal 0 V bias (inset), FIG. 2d is a histogram of the on/off ratio and mobility of the top-gate MoS2 TFT, where the on/off ratio is defined by current values under gate biases of 2.5 V and −7.5 V, respectively, FIG. 2e is the switching characteristics of the multiplexing TFT (T1) for row addressing, where the switching circuit periodically applies ‘ON’ (2.5 V) and ‘OFF’ (−7.5 V) voltages for 100 ms, FIG. 2f is the output response of the sensing TFT to an input 400 Hz sine wave and the sampling frequency of the readout circuit is 2.1 kilo-samples per second (kS s−1), FIG. 2g is a diagram of the device and its equivalent circuit for frequency response experiments, FIG. 2h shows the values of external contact impedance (Zcont) and internal impedance (Zin) according to the frequency of the signal applied to the electrolyte, and FIG. 2i is the current gain of the MoS2 active TFT over a wide frequency bandwidth (0.1-1000 Hz), showing a stable gain of 0.985±0.027 without drift tendency.

Referring to FIG. 2a, the characteristics of a test module in which each unit cell includes dual TFTs in series for embedded multiplexing (T1) and sensing (T2) were analyzed.

In particular, referring to FIG. 2b, the Id-Vg curve clearly shows the performance of the LT-MoS2 based TFT in depletion mode with a negative threshold voltage (Vth) of −1.4±0.6 V achieved by controlling sulfur vacancies.

Referring to FIG. 2c, the extracted transconductance (gm) demonstrated sufficient transduction capability of the active array at a 0 V bias, which converts electrophysiological potentials into drain-source currents. Thus, the high-throughput state facilitated the extraction of ECoG signals from the MoS2-based active array in a natural intracranial environment without additional voltage bias coupling or noise.

Furthermore, statistical results show that the MoS2 TFT array possesses adequate on-current, high switching performance, and excellent uniformity.

Referring to FIG. 2d, as shown in the histogram, the MoS2 TFT had an average mobility of 15.5±2.7 cm2 V−1 s−1 and a high ON/OFF ratio of ~108, which are essential prerequisites of ECoG arrays for high-speed multiplexing, high-density sampling, and low crosstalk noise.

Referring to FIG. 2e, in the two-transistor series structure, the switching characteristics for row addressing and the response to AC voltage signals were analyzed as fundamental indicators of an ideal active array. Measuring the response to an external square wave applied from the ROC revealed an ultra-low latency (less than 50 μs per row), combining both array and ROC switching and sampling delays. This allowed the temporal resolution of each unit in the 10×10 ECoG array to reach the 100-microsecond level (500 μs) in data reading, corresponding to a 2000 Hz sampling frequency, enabling the array to capture high spatiotemporal resolution of neural activity unprecedentedly. More importantly, the active array successfully reached an on-site switching level of ~73.9 dB within the same column. This was not only sufficient to suppress signal crosstalk but also helped in noise suppression of the device.

Referring to FIG. 2f, taking advantage of the aforementioned factors, it was confirmed that the output response of the ECoG sensor highly matched the input 400 Hz sine wave with millisecond accuracy and no output signal delay, indicating promising signal processing fidelity in the active matrix.

Referring to FIGS. 2g to 2h, the frequency-dependent response of the MoS2-based active array was another intrinsic aspect affecting the signal-to-noise ratio (SNR) and the fidelity of the output signal. According to the diagram of the device and its equivalent circuit (FIG. 2g), relevant elements are denoted as the external contact impedance (Zcont) and the input impedance (Zin) of the sensing TFT. The former exhibits an inverse nonlinear correlation of impedance with frequency, considering the equivalent impedance revealed by the capacitive electrolyte-electrode interface and the current I(f) flowing through the sensing gate and the dielectric layer (FIG. 2h). Meanwhile, the active TFT has a typical source-follower configuration for high input impedance (Zin), exhibiting excellent reliability due to the protection of the dielectric layer, which is hardly affected by the external environment (Zin=±2.5%). Here, in the active array, I(f) flowing through the Al2O3 insulator was limited (~10-11 A), showing minimal distortion compared to passive arrays. Consequently, a stable gain of 0.958±0.027 (ideal value=1) was obtained, proving that the output amplitude remains nearly constant over a bandwidth of 0.1-1000 Hz and the fidelity of ECoG signals was secured by the linear frequency response.

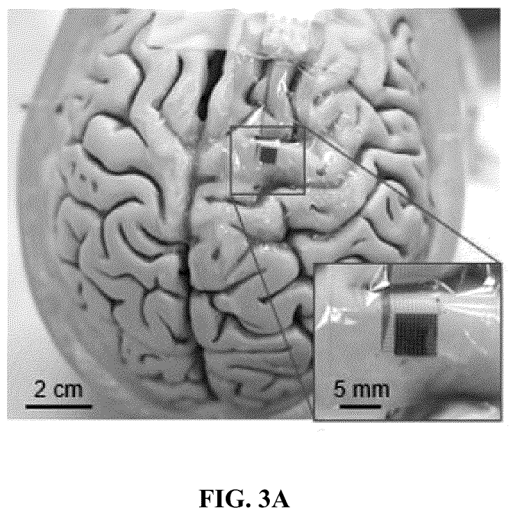

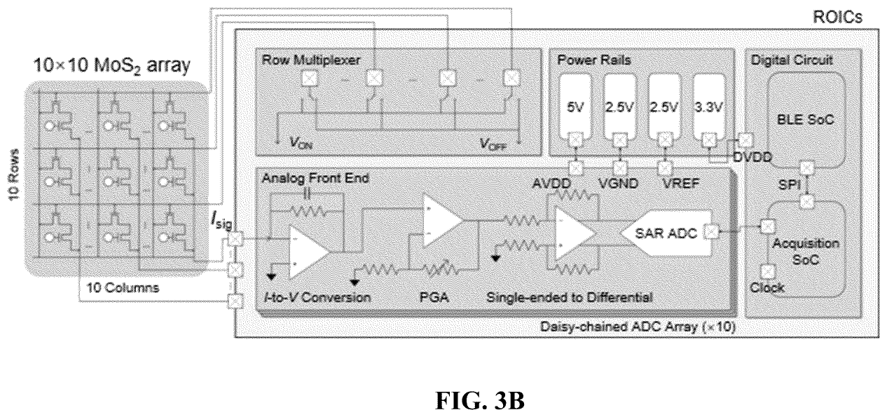

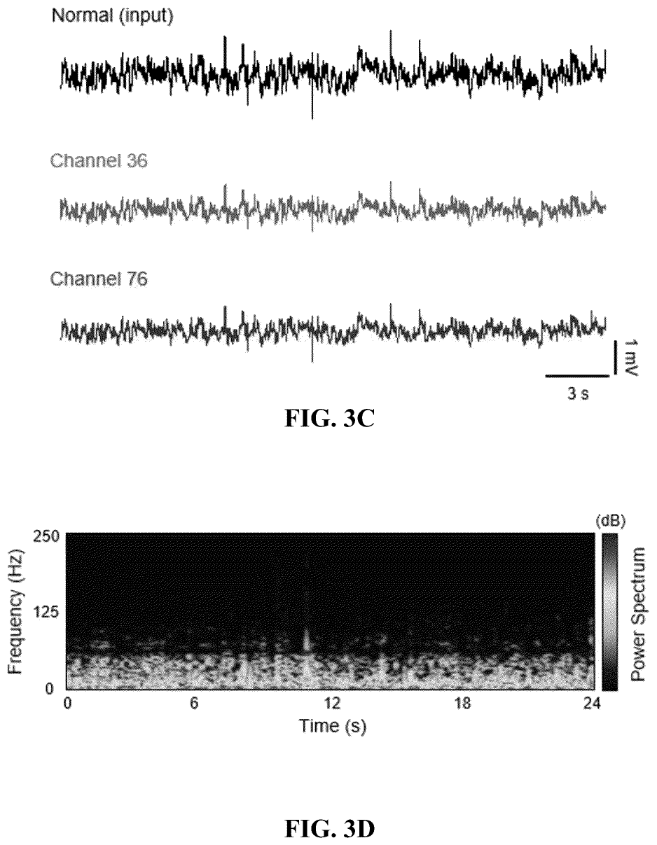

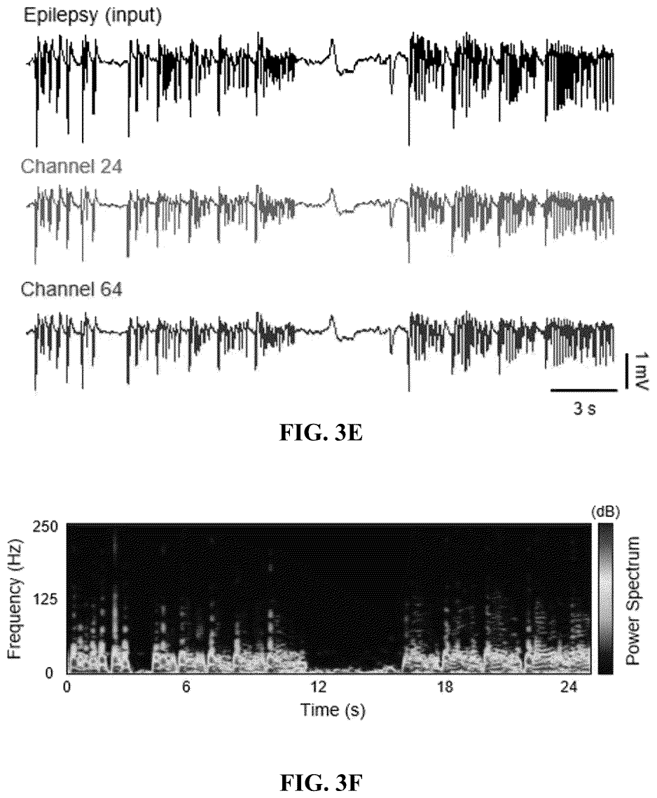

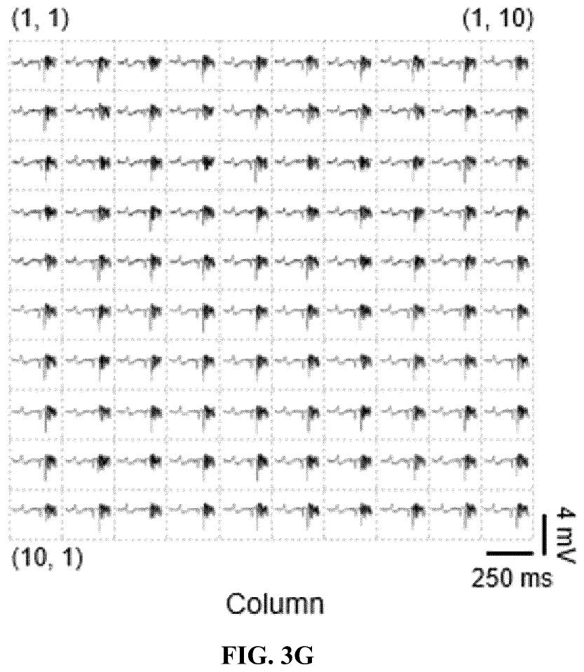

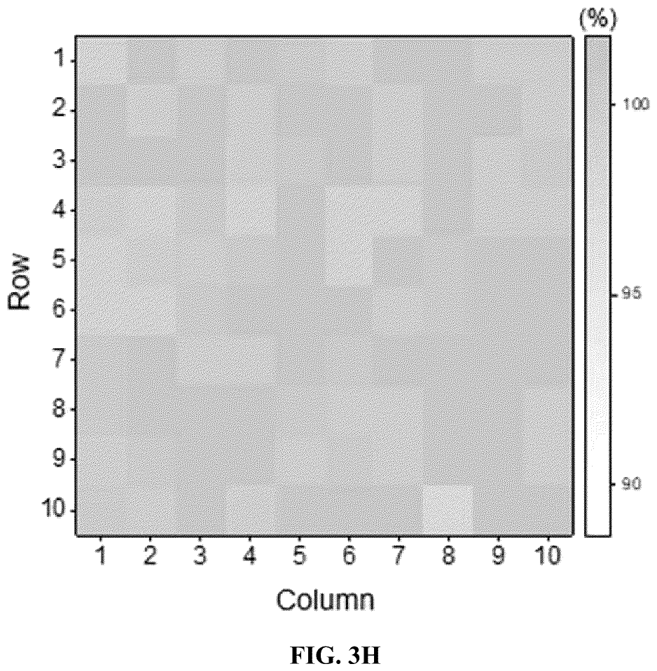

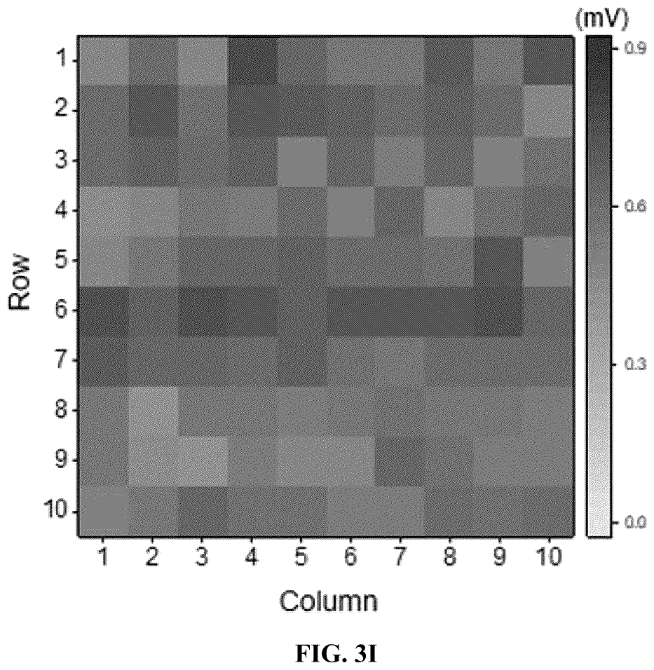

FIG. 3 shows in vitro recordings of normal and epileptic activity with high spatiotemporal resolution, according to an embodiment of the present invention.

Specifically, FIG. 3a is a brain-like setup of a low-impedance neural interface, FIG. 3b is a schematic diagram of a customized readout circuit (ROC), FIG. 3c is a recording of normal physiological activity from representative channels (No. 36 and No. 76), FIG. 3d is a time-frequency spectrum of the physiological activity of channel 36 without abnormal paradoxical discharges, FIG. 3e is epileptic spike activity captured by MoS2 array channels (No. 24 and No. 64), FIG. 3f is a time-frequency spectrum of ictal-like epileptic discharges, FIG. 3g is the spatial distribution of ECoG potential signals in a 10×10 array, FIG. 3h is a time-skewed color mapping of a 10×10 array calibrated to the first pre-ictal-like spike, and FIG. 3i is an amplitude-skewed color mapping of a 10×10 array calibrated to the first pre-ictal-like spike.

Referring to FIG. 3a, to investigate the reliability and practicality of the MoS2-based active array and the customized ROC, an in vitro cortical model mimicking ECoG signals was constructed. To create an ideal artificial neural interface, the surface of the cortical model was covered with a low-impedance NaCl gel, and the surface was kept sufficiently wet with phosphate-buffered saline (PBS, pH=7.4). In addition, to input consistent ECoG signals, an Ag/AgCl reference electrode was deeply embedded in the gel and connected to a signal generator. The corresponding ROC was designed to allow fast switching and sampling of up to 2 kHz. Current-mode signals were converted into voltage-mode signals through a trans-impedance circuit, which were then sampled and wirelessly transmitted via Bluetooth.

Referring to FIGS. 3c to 3f, both normal physiological activity and epileptic pathological activity were observed through an in vitro neural interface where the input ECoG signals used were collected by a reference graphene-based passive electrode. In FIG. 3c, the normal physiological activity of two representative channels appeared with high fidelity. Furthermore, the time-frequency spectrum extracted from channel 36 showed the presence of continuous and stable neural activity throughout the recording without abnormal paradoxical discharges (FIG. 3d). In sharp contrast, the epileptic activity in FIG. 3e exhibited ictal-like spikes with short inter-spike durations. FIG. 3f shows a clear difference between normal physiological activity and epileptic discharges, the latter exhibiting higher power intensity in a bursting discharging pattern. As in real brain signals, the device showed a correlation between output/input signals (94.4%), demonstrating the excellent SNR, linear responsiveness, and temporal resolution of the MoS2 device.

Referring to FIGS. 3g to 3i, to further investigate the performance uniformity of the 100-channel MoS2-based array, the spatial distribution of ECoG potentials is provided in FIG. 3g. Since the purpose of the in vitro mapping test was to explore the time and amplitude progression of potentials between different pixels of the array, time calibration was specially tailored to eliminate system errors occurring in the input electrode geometry and row-wise scanning mode. The spatial color mapping in FIG. 3h reveals imperceptible time differences in milliseconds, with a maximum delay time normalized to 130 ms being only 5%. Amplitude differences were extracted by the first pre-ictal-like spikes, and a reasonable amplitude variation of ±20% was obtained (FIG. 3i). This was in good agreement with the statistical results for the field-effect mobility of the array, as the amplitude of the output signal is usually proportional to the current level of the channel at 0 V gate bias.

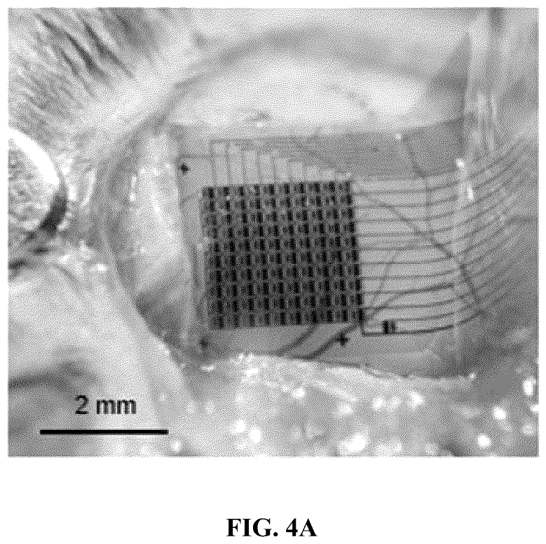

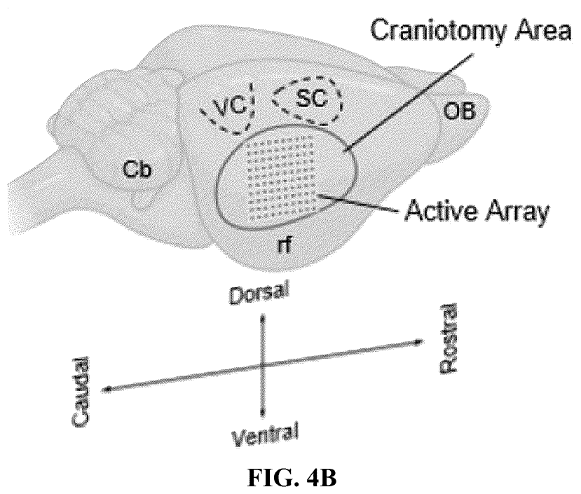

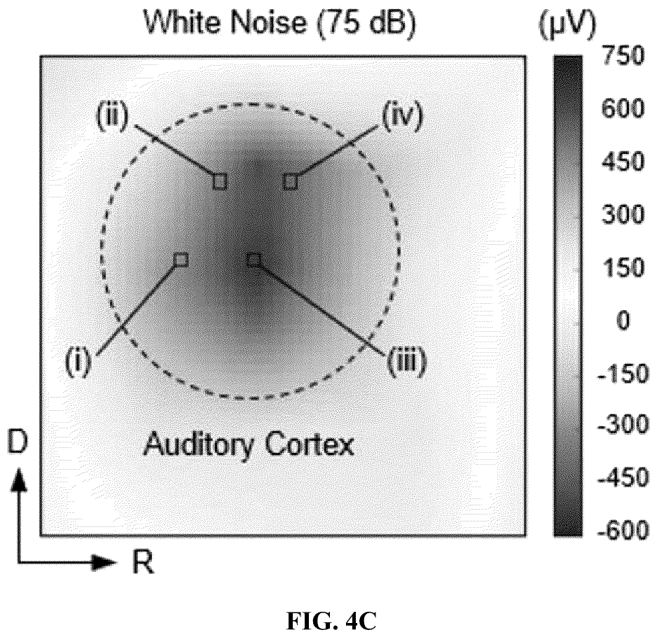

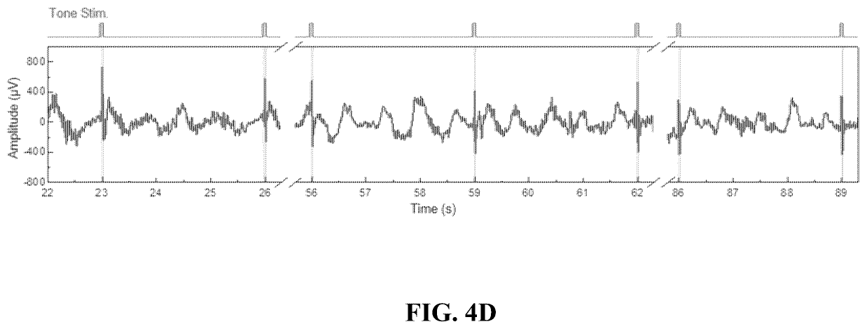

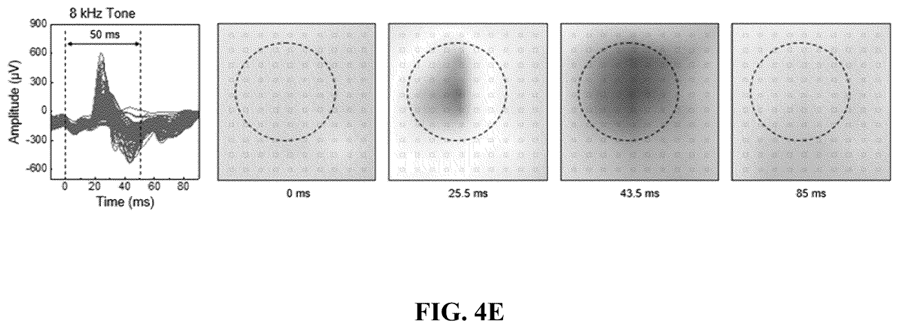

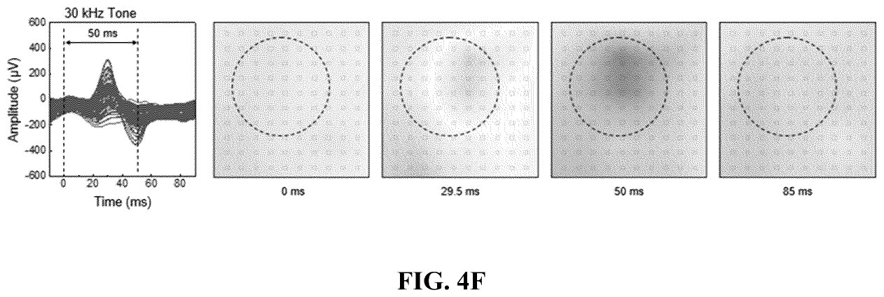

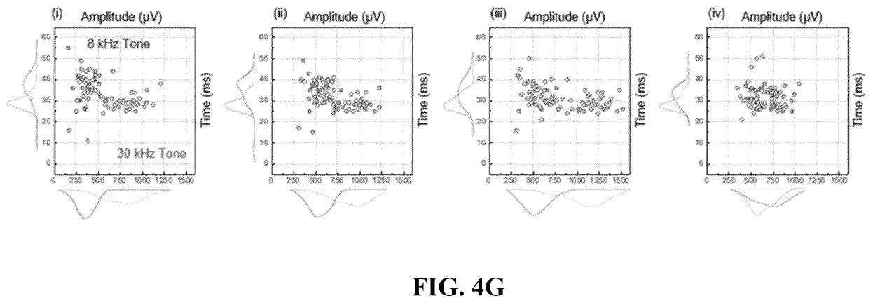

FIG. 4 shows in vivo monitoring of event-related potentials (ERPs) of the auditory cortex induced by acoustic stimuli, according to an embodiment of the present invention.

Specifically, FIG. 4a is an optical image of an ECoG active array placed over the auditory cortex of a mouse, FIG. 4b is a schematic diagram of the spatial location of the device recording site in the right cerebral hemisphere, FIG. 4c is spatial information of the auditory cortex extracted through ECoG activity responding to regular 50-ms white noise stimuli, FIG. 4d is a continuous recording of auditory responses induced by white noise from a channel, along with a paradigm of time-calibrated tone click stimuli (red), FIG. 4e is an example of a 100-ms ERP induced by an 8 kHz tone stimulus and corresponding spatial mapping of ERP distribution through four frames in chronological order, FIG. 4f is the ERP and matching spatiotemporal neural activity pattern of a 30 kHz pure tone stimulus, and FIG. 4g is a scatter plot showing ERP characteristics in terms of amplitude (x-axis) and time delay (y-axis) of spikes extracted from localized positions (i)-(iv) of the auditory cortex (FIG. 4c), where Gaussian distributions of ERP amplitude and delay time (inset) are presented for normality tests for one-way analysis of variance (ANOVA).

Referring to FIGS. 4a to 4c, next, it was investigated whether the ECoG active array effectively captures neural responses to auditory stimuli in an anesthetized live animal. The active array (2.5×2.5 mm²) was scaled down and inserted into an implantable area under a craniotomy of a small mouse brain (FIG. 4a) and placed on the surface of the auditory cortex (FIG. 4b). Compared to high-density silicon-based ECoG arrays for in vivo recording with column sizes of 500-600 μm, this array, with a column size of about 250 μm and a 4-fold higher mapping resolution of 16 pixels mm², was able to profile neural activity in response to white noise stimuli in the auditory cortex in detail (1.4×1.4 mm²) (FIG. 4c).

Furthermore, referring to FIGS. 4d to 4g, the high resolution allowed for the identification of subtle differences in acoustic response patterns to various pure tone stimuli, facilitating subsequent investigations into auditory mismatch negativity (AMMN) and tonotopic mapping. For example, thanks to stable long-term recording with a time-calibrated stimulus paradigm (FIGS. 4d), 100-ms ERPs induced by 8 kHz and 30 kHz tone pips (75 dB, 50-ms duration) could be extracted (FIGS. 4e, 4f). Due to the high spatiotemporal fidelity of the array, subtle differences in auditory responses corresponding to different pure-tone acoustic stimuli could be mapped; the auditory cortex exhibited a response amplitude of 1.1 mV for the 8 kHz stimulus at an elapsed time of 25.5 ms, which is similar to the 0.7 mV for the 30 kHz-induced ERP at a time of 29.5 ms. This result showed that the ECoG active array can accurately map and distinguish subtle details of dynamic ERP patterns induced by different stimuli across the rostrocaudal axis. Scatter plots sampled at four different positions along the rostrocaudal axis further showed the existence of distinct characteristics of auditory responses specific to different local regions of the auditory cortex (FIG. 4g). At positions (i)-(iii) randomly extracted from ECoG recording samples, ERPs induced by two different tone clicks showed significant distribution differences (N=40, t-test), and the 8 kHz-induced ERP indicated a higher statistical probability of higher spike amplitude and lower response latency (p***<0.001). However, as the position gradually moved toward the rostral side, an abrupt characteristic transition of the ERP pattern was observed at position (iv), where a more responsive ERP was induced by the 30 kHz tone stimulus rather than the 8 kHz tone (p**<0.01).

To enable the construction of tonotopically-arranged auditory maps allowed only by high-resolution mapping tools, the ECoG array was mounted on the cortical surface, and frequency-specific responses to pure tone pips (50 ms, 2-ms cosine square ramp) of 80 frequencies and 8 SPLs (0-70 dB, 10-dB steps) were accessed via an in-ear speaker. A frequency map was constructed by measuring local field potentials (LFPs) for random combinations of sound frequency (range: 4-32 kHz with 0.1-octave intervals) and intensity (range: 0-70 dB) to calculate characteristic frequency (CF). To define the CF for constructing a tonotopic map of the primary auditory cortex (A1), the tip frequency of a V-shaped tuning curve, which describes the minimum acoustic intensity that triggers an LFP response, was selected. Various tonotopic map features were characterized, including receptive field (RF) size, tuning curve size, firing rate, spike amplitude, and bandwidth (BW) 20 (20 dB above the tip). These results showed that the ECoG active array evaluates frequency maps with unprecedented spatiotemporal resolution.

What is claimed is:

1. An active array for a high-resolution active cortical signal measurement device, comprising:
a unit pixel including a sensing TFT for sensing a brain signal of a living body, and a multiplexing TFT connected in series with the sensing TFT for multiplexing.

2. The active array of claim 1,
wherein the sensing TFT and the multiplexing TFT include two-dimensional (2D) molybdenum disulfide (MoS2) thin-film transistors,
high-resolution active array for cortical signal measurement device

3. The active array of claim 1,
wherein a size of a column of the unit pixel is 1000 μm or less,
high-resolution active array for cortical signal measurement device.

4. The active array of claim 1,
wherein a resolution in the active array is 1 pixel/mm² or more,
high-resolution active array for cortical signal measurement device.

5. A method of manufacturing an active array for a high-resolution active cortical signal measurement device, comprising:
depositing a buffer layer on a polymer substrate (S100);
forming a two-dimensional (2D) transition metal dichalcogenide channel on the buffer layer (S200);
forming a source electrode and a drain electrode on the two-dimensional transition metal dichalcogenide channel (S300);
forming a gate dielectric layer on the source electrode and the drain electrode (S400);
forming a gate electrode on the gate dielectric layer (S500); and
forming an encapsulation layer on the gate electrode (S600),
the method of manufacturing an active array for a high-resolution active cortical signal measurement device.

6. The method of claim 5,
wherein the two-dimensional transition metal dichalcogenide is at least one selected from the group consisting of MoS2, WSe2, WS2, and MoTe2,
the method of manufacturing an active array for a high-resolution active cortical signal measurement device.

7. The method of claim 5,
wherein the step (S200) of forming the two-dimensional transition metal dichalcogenide channel on the buffer layer,
includes directly growing the two-dimensional transition metal dichalcogenide on the buffer layer at a temperature ranging from 100° C. to 400° C. (S210),
the method of manufacturing an active array for a high-resolution active cortical signal measurement device.

8. The method of claim 5,
wherein the step (S200) of forming the two-dimensional transition metal dichalcogenide channel on the buffer layer,
includes growing the two-dimensional transition metal dichalcogenide at a temperature exceeding 400° C. and then transferring the same onto the buffer layer (S220),
the method of manufacturing an active array for a high-resolution active cortical signal measurement device.

9. The method of claim 6,
wherein the two-dimensional transition metal dichalcogenide is two-dimensional molybdenum disulfide (MoS2), and
the step (S210) of directly growing the two-dimensional molybdenum disulfide on the buffer layer at a temperature ranging from 100° C. to 400° C.,
uses molybdenum hexacarbonyl (MHC) and anhydrous dimethyl sulfide (DMS) as precursors,
the method of manufacturing an active array for a high-resolution active cortical signal measurement device.

10. The method of claim 9,
wherein the molybdenum hexacarbonyl (MHC) and the anhydrous dimethyl sulfide (DMS) are provided to metal-organic chemical vapor deposition (MOCVD) in a form of a mixed precursor solution,
the method of manufacturing an active array for a high-resolution active cortical signal measurement device.
